(19) United States
(12) Patent Application Publication
(10) Pub. No.: US 2021/0330753 A1
(43) Pub. Date: Oct. 28, 2021

(54) METHODS OF TREATING COVID-19 MEDIATED LUNG DAMAGE USING SURFACTANTS AND NATURAL ANTIBODIES

(71) Applicants: Ghassan S Kassab, La Jolla, CA (US); Carlos A Labarrere, San Diego, CA (US)

(72) Inventors: Ghassan S Kassab, La Jolla, CA (US); Carlos A Labarrere, San Diego, CA (US)

(21) Appl. No.: 17/242,142

(22) Filed: Apr. 27, 2021

Related U.S. Application Data
(60) Provisional application No. 63/016,227, filed on Apr. 27, 2020.

(51) Int. Cl. A61K 38/17; C07K 16/42; A61P 31/14

(52) U.S. Cl. CPC A61K 38/395; A61K 2039/505; A61P 31/14; C07K 16/42

(57) ABSTRACT

Disclosed is a method of treating respiratory viruses including coronaviruses such as SARS-CoV-2 using surfactant, surfactant protein A and/or surfactant protein D. The surfactant and surfactant protein treatment can be used in combination with immunoglobulin M administered intravenously. Surfactant proteins used in these embodiments, especially surfactant protein D, can be harvested from an exogenous source such as pigs, as they show improved resilience against viruses.

### PRIORITY

The present patent application is related to, and claims the priority benefit of, U.S. Provisional Patent Application Ser. No. 63/016,227, filed on Apr. 27, 2020, the contents of which are hereby incorporated by reference in their entirety into this disclosure.

### BACKGROUND

Coronavirus disease 2019 (COVID-19) is caused by the severe acute respiratory syndrome coronavirus 2 (SARS-CoV-2). It is a positive-sense single-stranded RNA virus named for the crown-like spikes on its surface (S-protein) that allows the virus to enter the host cells. This family of viruses mutates easily and infects animals and humans. COVID-19 affects the lower respiratory tract that lines the whole pulmonary tree; mainly alveoli where the exchange of oxygen and carbon dioxide occurs during respiration, causing respiratory distress attributed to alveolar damage associated with severe immunopathological lesions; which is the most common cause of death. Patients initially develop flu-like symptoms and can progress to shortness of breath and complications from pneumonia establishing the need for a respirator. People of all ages can be infected, but the risk of severe disease and death is highest for older people, people having heart disease, chronic lung disease, diabetes and cancer. As the virus enters the lung cells, it starts replicating, our body recognizes the viruses as foreign invaders triggering an immune response to control them and stop replication.

The immune response to COVID-19 can also damage lung tissues, however, through severe inflammation complicating pneumonia. Pattern recognition proteins (PRPs) that are components of surfactant, like surfactant protein D (SP-D) and surfactant protein (SP-A), bind influenza A RNA viruses (IAV) inhibiting attachment and entry of the virus and also contribute to enhanced clearance of SP-opsonized virus via interactions with phagocytic cells. Another PRP, Immunoglobulin M natural antibodies (IgM NAbs) enhance late apoptotic cell clearance in the lungs by alveolar macrophages.

In view of the same, a treatment that uses a surfactant and SP-D as antiviral therapies administered by inhalation and/or after tracheal intubation in patients requiring ventilators can provide acute protection against invading IAV particles with little toxicity and high tolerance would be appreciated in the medical arts.

### BRIEF SUMMARY

The present disclosure includes disclosure of intravenous (i.v.) administration of IgM NAbs to enhance antiviral protection and late apoptotic cell clearance in the lungs by alveolar macrophages. The present disclosure includes discussion of the efforts to identify the effects of surfactant and SP-D on human alveolar type II cells infected with coronavirus in vitro, and to identify the effects of surfactant, SP-D, IgM NAbs and their combination upon alveolar damage in an infected swine model. Said treatments can dramatically reduce the need of ventilation and speed up the recovery of patients affected by COVID-19 viral infection.

The present disclosure can be applied to the treatment of other severe acute respiratory syndrome coronaviruses including is SARS-CoV-2 and its mutations.

In one embodiment, a method of treating a mammalian patient infected with a respiratory virus comprises the step of administering a therapeutically effective amount of surfactant.

In one embodiment, a method of treating a mammalian patient infected with a respiratory virus comprises the step of administering a therapeutically effective amount of surfactant and surfactant protein.

In one embodiment, a method of treating a mammalian patient infected with a respiratory virus comprises the step of administering a therapeutically effective amount of surfactant protein. In an alternate embodiment the surfactant protein comprises surfactant protein D. In another embodiment the surfactant protein comprises surfactant protein A. In a further embodiment, the surfactant protein comprises a combination of SP-D and SP-A.

Either or both of SP-D and SP-A can be exogenous and are preferably derived from a porcine source.

The surfactant and surfactant protein are preferably introduced into the airways of the patient and administered by inhalation and travel to the alveoli.

The embodiments of administering surfactant and/or surfactant proteins can also be combined with the administering of a therapeutically effective amount of Immunoglobulin M natural antibodies (IgM NAbs). IgM NAbs is preferably administered intravenously.

A method of treating a human patient infected with a severe acute respiratory syndrome coronavirus or a variant thereof, comprising the step of administering a therapeutically effective amount of surfactant and porcine SP-D and SPD-A.

In another embodiment a human patient infected with a severe acute respiratory syndrome coronavirus or a variant thereof, is treated by of administering a therapeutically effective amount of SP-D. In an alternate embodiment, the method of treatment includes a further step of administering a therapeutically effective amount of IgM NAbs.

In another embodiment a human patient infected with a severe acute respiratory syndrome coronavirus or a variant thereof, is treated by of administering a therapeutically effective amount of porcine derived SP-D and a therapeutically effective amount of IgM NAbs.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows alveolus depicting how inhaled administration of surfactant (containing SP-D and SP-A) and/or SP-D (blue arrow) facilitates viral clearance by alveolar macrophages. Surfactant produced by alveolar type II cells contributes with virus removal. Pattern recognition proteins (IgM, CRP, SP-D, SP-A) also contribute with apoptotic cell removal through pattern recognition protein receptors (PRCPs) on alveolar macrophages. IgM from circulation contributes with the removal. Abbreviations: LysoPC, lysophosphatidylcholine.

FIG. 2 shows Natural antibodies (NAbs) are removed by Phosphorylcholine (PC) and C-reactive protein (CRP) but not albumin (A); and displaced from myocardial capillaries (B) being found in the eluates following incubation (C).

FIG. 3 shows High levels of IgM natural antibodies (IgM NAbs) in myocardial capillaries (orange bars) and serum (blue bars) associate with reduced inflammation (as measured by serum CRP), less CAV, CAV severity, MACE and death due to CAV.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Coronavirus disease 2019 (COVID-19) is caused by the severe acute respiratory syndrome coronavirus 2 (SARS-CoV-2). It is a positive-sense single-stranded RNA virus named for the crown-like spikes on its surface. This family of viruses mutates easily and infects mostly bats, pigs, small mammals and humans. Recently, they have become growing players in infectious-disease outbreaks world-wide. Several strains are known to infect humans, including COVID-19, which affects the lower respiratory tract that lines the whole pulmonary tree; mainly alveoli where the exchange of oxygen and carbon dioxide occurs during respiration, causing respiratory distress attributed to alveolar damage associated with immunopathological lesions; which is the most common cause of death. Patients initially develop flu-like symptoms and can progress to shortness of breath and complications from pneumonia establishing the need for a respirator. People of all ages have been infected, but the risk of severe disease and death is highest for older people, people having heart disease, chronic lung disease, diabetes and cancer. As the virus enters the lung cells, it starts replicating. Our body recognizes all viruses as foreign invaders triggering an immune response to control them and stop replication. The immune response to COVID-19 can also damage lung tissues through severe inflammation complicating pneumonia. Pneumonia causes that alveoli become inflamed and filled with fluid, making it harder to breathe and deliver oxygen to blood, potentially triggering a cascade of respiratory/cardiac complications. Lack of oxygen leads to more inflammation, and body complications resulting in severe liver and kidney damage, and patient's death. Patients must be placed on ventilators for weeks as they recover from the viral infection. It is projected that the number of patients requiring respirators surpasses the number of ventilators presently available in hospitals and ICUs, making urgent the need for avoiding reaching the need for ventilators and/or promptly recover from the lung infection.

The number of COVID-19 confirmed cases reported to WHO continues to raise exponentially worldwide²⁹. During the past 2 decades, several viral epidemics, among them the severe acute respiratory syndrome coronavirus (SARS-CoV), the H1N1 influenza, the Middle East respiratory syndrome coronavirus (MERS-CoV), and now the new COVID-19 have shown all to be lethal. As of Apr. 24, 2020, COVID-19 has caused 181938 deaths globally out of 2626321 confirmed cases reported in 212 countries. Presently, no specific treatment for COVID-19 exists. The principal clinical management for this lethal disease is fundamentally a symptomatic treatment with intensive care organ support for seriously ill patients. All world organizations, including the WHO have mainly focused on avoiding transmission, implementing infection control measures and performing screen controls in travelers throughout the world. At time of initial writing, no vaccines presently exist although immediate funding was made available to develop them. As it occurred for SARS-CoV and MERS-CoV more support for developing treatments to reduce mortality and/or treat or prevent COVID-19 disease are needed²⁵. There is an urgent need for funding directed to advancing novel therapies to avoid severe coronavirus infection, since development of severe acute respiratory distress syndrome associated with severe lung pathology leads to death, and patients who survive intensive care-associated excessive inflammation develop long-term lung damage and fibrosis causing functional disability and reduced quality of life²⁵⁻²⁷.

According to the World Health Organization (WHO), viral diseases continue to emerge and represent a serious issue to public health. The Spanish flu, also known as the 1918 flu (H1N1) pandemic, and in the last twenty years, several viral epidemics such as the severe acute respiratory syndrome coronavirus (SARS-CoV) in 2002 to 2003, and H1N1 influenza in 2009, have been recorded¹¹. Most recently, the Middle East respiratory syndrome coronavirus (MERS-CoV) was first identified in Saudi Arabia in 2012¹¹. At present, an epidemic of cases with unexplained low respiratory infections detected in Wuhan, the largest metropolitan area in China's Hubei province, was first reported to the WHO Country Office in China, on Dec. 31, 2019¹¹. This is actually known as the coronavirus disease 2019 (COVID-19) caused by the severe acute respiratory syndrome coronavirus 2 (SARS-CoV-2). Coronaviruses (CoVs), a large family of single-stranded RNA viruses, can infect animals and humans, causing respiratory, gastrointestinal, hepatic, and neurologic diseases^{12,13}. As the largest known RNA viruses, CoVs are divided into four genera: Alpha-, beta-, gamma- and delta-coronavirus^{13,14}. To date, there have been identified six human coronaviruses (HCoVs), including the alpha-CoVs HCoVs-NL63 and HCoVs-229E and the beta-CoVs HCoVs-OC43, HCoVs-HKU1, severe acute respiratory syndrome-CoV (SARS-CoV)¹⁵, and Middle East respiratory syndrome-CoV (MERS-CoV)¹⁶. New coronaviruses emerge periodically in humans, mainly due to the high prevalence and wide distribution of coronaviruses, the large genetic diversity and frequent recombination of their genomes, and the extended human-animal interface activities^{17,18}. On 30 Jan. 2020, the World Health Organization (WHO) declared that CoVID-19 is a “public-health emergency of international concern”¹⁹. Similar to patients with SARS-CoV and MERS-CoV, some patients with the novel coronavirus (2019-nCoV) COVID-19 develop acute respiratory distress syndrome (ARDS) with characteristic pulmonary ground glass changes on imaging. In most moribund patients, COVID-19 infection is also associated with an inflammation-associated cytokine storm²⁰⁻²⁴. In patients who survive intensive care, these aberrant and excessive immune responses lead to long-term lung damage and fibrosis, causing functional disability and reduced quality of life²⁵⁻²⁷. The pandemic is escalating rapidly where COVID-19 affects the lower respiratory tract causing respiratory distress, the most common cause of death due to alveolar damage. Due to the possibility that the number of patients in need for ventilation can surpass the number of available respirators, and to the high death toll with severe disease, there is an urgent need to enhance the innate pulmonary immune response.

At present, there is no vaccine or antiviral treatment for human and animal coronavirus, so that identifying the drug treatment options as soon as possible is critical for the response to the CoVID-19 outbreak. WHO has announced that a vaccine for SARS-CoV-2 should be available in 18 months, but achieving this will require funding and public interest to be maintained even if the threat level falls^{13,28.} The principal clinical management is largely symptomatic treatment, with organ support in intensive care for seriously ill patients²⁵. WHO and other global public health bodies have mainly focused on preventing transmission, infection control measures, and travelers' screenings. The development of vaccines has received immediate funding; however, as with SARS-CoV and MERSCoV, support for developing treatments for 2019-nCoV that reduce mortality has not been forthcoming. There is an urgent need for focusing funding and scientific investments into advancing novel therapeutic interventions for coronavirus infections. All three coronaviruses induce excessive and aberrant non-effective host immune responses that are associated with severe lung pathology, leading to death.

Scientists have demonstrated that components of surfactant, a complex mixture of phospholipids (PL) and proteins (SP) that reduce surface tension at the air-liquid interface of the alveolus, is made up of 70-80% PL, 10% SP-A, B, C and D, and 10% neutral lipids^{1,2}. It has been demonstrated that SP-D and SP-A, two pattern recognition proteins (PRPs) of the innate immune system³, bind influenza A RNA viruses (IAV) inhibiting attachment and entry of the virus and also contribute to enhanced clearance of SP-opsonized virus via interactions with phagocytic cells^{4,5}. Another PRP, IgM natural antibodies (IgM NAbs) enhance late apoptotic cell clearance in the lungs by alveolar macrophages⁶. In addition, SP-D modulates the inflammatory response and helps maintain an equilibrium between effective neutralization/killing of IAV, and protection against alveolar damage resulting from IAV-induced excessive inflammatory responses. SP-D from pigs exhibits distinct anti-IAV properties neutralizing a broad range of IAV and wild-type porcine SP-D exhibits strong antiviral properties against a much broader range of IAV strains/subtypes compared to human SP-D as it is naturally expressed in the airways⁴. It has been demonstrated that primary human alveolar type II cells infected with SARS-CoV, maintained under air-liquid conditions, can generate a vigorous innate immune response⁷, and different cell culture systems are available to recapitulate the human airways, including the air-liquid interface human airway epithelium model that can be used to identify antivirals, evaluate compound toxicity and viral inhibition⁸.

The use of surfactant, SP-D, IgM NAbs and their combination as antiviral therapies, earlier in patients at risk or infected by aerosol spray administration, and directly in patients on ventilators, is disclosed in detail herein. Pulmonary surfactant and SP-D administration will provide acute protection against COVID-19, and i.v. administration of IgM NAbs will enhance antiviral protection and late apoptotic cell clearance. Since SP-D is a naturally occurring substance in the airways, we anticipate little toxic effects and a relatively high immunogenic tolerance in humans.

This disclosure describes the protective effect of surfactant, and SP-D upon COVID-19 pulmonary infection following SP-D-mediated virus binding and inhibition of the attachment and entry of the virus contributing to enhanced clearance of SP-D-opsonized virus via interactions with phagocytic cells⁴. The use of SP-D as an antiviral therapy offers several advantages. First, SP-D and especially porcine SP-D neutralize a broad range of IAVs and it is unlikely that a single genome IAV mutation would induce resistance against SP-D antiviral activity. Second, SP-D can be administered into the airways to provide acute protection against invading IAV particles. Third, since SP-D naturally occurs in the airways, little toxic effects and high immunogenic tolerance are expected for SP-D therapy in humans. Finally, the combination of surfactant, SP-D and IgM NAbs will amplify antiviral neutralization and removal in infected lungs. The research disclosed herein is innovative because it focuses on understanding the protective effect of soluble innate immunity on COVID-19. The novel feature of this research lies in its potential to open a fundamentally new clinical approach to treatment, prevention and management of the current COVID-19 infection crisis.

The present disclosure includes disclosure of using a surfactant and SP-D as antiviral therapies administered by inhalation and/or after tracheal intubation in patients requiring ventilators. Using surfactant and SP-D as antivirals would offer several advantages. SP-D neutralizes a broad range of IAVs and it is unlikely that a single genome IAV mutation would induce resistance against SP-D antiviral activity. Inhaled/intratracheal SP-D can provide acute protection against invading IAV particles. Since SP-D is in surfactant, little toxicity and a relatively high immunogenic SP-D tolerance are anticipated in humans. Intravascular (i.v.) administration of IgM NAbs will enhance antiviral protection and late apoptotic cell clearance in the lungs by alveolar macrophages. Specifically, the following items are discussed herein: 1) the identification of the effects of surfactant and SP-D on human alveolar type II cells infected with coronavirus in vitro (with in vitro studies providing data on the antiviral effects of SP-D in alveolar type II cells, evaluation of variations in proinflammatory cytokine and chemokine release and variability in expression of angiotensin converting enzyme 2, the COVID-19 receptor^{9,10}.), and 2) the identification of the effects of surfactant, SP-D, IgM NAbs and their combination upon alveolar damage in an infected swine model, which provides evidence for the efficacy of inhaled surfactant and SP-D, and the administration of IgM NAbs and its effects upon alveolar inflammation. As noted herein, a positive effect of surfactant and PRPs would reduce need for ventilation. Avoiding the need for ventilation can dramatically impact the healthcare system and speed up the recovery of patients affected by COVID-19 viral infection.

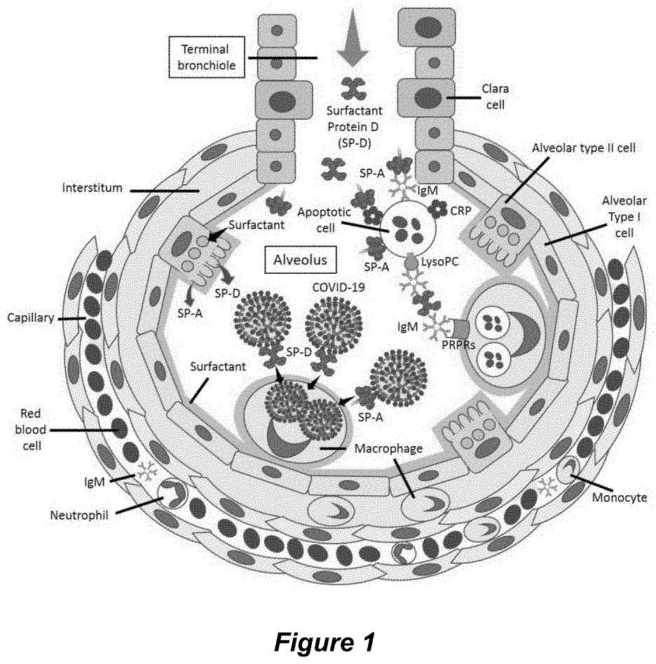

In addition, another RNA virus, the influenza A virus (IAV) is a major cause of respiratory tract infections resulting in a highly contagious disease leading to excess morbidity and mortality every year. Nonspecific innate immune mechanisms play a key role in protection against viral invasion at early stages of infection⁴. Surfactant protein D (SP-D), a soluble protein present in mucosal secretions of the lung, is an important component of this initial barrier that helps to prevent and limit respiratory IAV infections⁴. SP-D binds IAVs inhibiting cell attachment and entry of the virus and contributes to enhanced clearance of SP-D-opsonized virus by phagocytic cells. SP-D helps maintaining a balance between effective IAV neutralization/killing, and protection against alveolar damage resulting from IAV-induced excessive inflammatory responses⁴. SARS-CoVs infect host cells with their surface glycosylated S-protein, and S-protein activates macrophages through angiotensin converting enzyme 2 (ACE2) receptor-binding. SP-D binds S-protein leading to virus killing regulating pulmonary inflammation³⁰. The usefulness of a surfactant therapy has been clearly demonstrated in neonates without complications³¹. Defective pulmonary surfactant metabolism results in respiratory distress with attendant morbidity and mortality³². Treatment with exogenous surfactant has saved the lives of thousands of premature babies in the past few decades revolutionizing the treatment of respiratory distress syndrome³³. This disclosure includes the use of surfactant (containing both SP-D and SP-A) and SP-D as antiviral drugs administered by inhalation and/or after tracheal intubation in patients at risk, sick or requiring ventilators to reach pulmonary alveoli (FIG. 1). The use of surfactant and SP-D as antiviral drugs would offer several advantages. SP-D and especially porcine SP-D⁴ neutralize a broad range of IAVs and it is unlikely that a single genome IAV mutation would induce resistance against the antiviral activity of SP-D. SP-D can be administered into the airways to provide acute protection against invading IAV particles. Since SP-D is naturally found in the airways, little toxic effects and a relatively high immunogenic tolerance for such a biotherapeutic treatment in humans are anticipated. Another pattern recognition protein, IgM natural antibodies (IgM NAbs) enhances pulmonary alveolar late apoptotic cell clearance⁶, and i.v. administration of IgM NAbs will intensify antiviral protection and late apoptotic cell removal in the lungs by alveolar macrophages.

A. Surfactant Replacement Therapy for Neonates with Respiratory Distress Syndrome.

Pulmonary surfactant is a secreted, extracellular complex of lipids and proteins, which lines the alveolar compartment at the external air/tissue interface, produced by alveolar type II cells (FIG. 1), that reduces surface tension at the air-liquid interface of the alveolus and plays an important role in regulating inflammatory processes within the lung^{1,2}. It is made up of about 70% to 80% PL, mainly dipalmitoylphosphatidylcholine, 10% SP-A, B, C and D, and 10% neutral lipids, mainly cholesterol. SP-A and SP-D are hydrophilic and participate in the innate host defense immune system¹. Respiratory failure due to surfactant deficiency is a major cause of morbimortality in preterm infants³³. Surfactant replacement therapy is a safe and effective way to treat immaturity-related surfactant deficiency³⁴. Surfactant administration in preterm infants with established respiratory distress syndrome (RDS) reduces mortality and lowers the risk of chronic lung disease³⁴. Surfactant therapy given as prophylaxis or rescue treatment allows the SP-D binding of RNA viruses like IAV leading to formation of SP-D/virus complexes can also result in distinct interactions with immune cells leading to enhanced phagocytosis and modulation of the inflammatory response⁴.

B. SP-D Treatment for RNA Viral Infections.

A soluble protein present in mucosal secretions of the lung, surfactant protein D (SP-D), is an important component of this initial barrier that helps to prevent and limit influenza A virus (IAV) infections of the respiratory epithelium^{3,4}. This collagenous C-type lectin binds IAVs and thereby inhibits attachment and entry of the virus but also contributes to enhanced clearance of SP-D-opsonized virus via interactions with phagocytic cells. In addition, SP-D modulates the inflammatory response and helps to maintain a balance between effective neutralization/killing of IAVs, and protection against alveolar damage resulting from IAV-induced excessive inflammatory responses. The mechanisms of interaction between SP-D and IAV not only depend on the structure and binding properties of SP-D but also on strain-specific features of IAV⁴. SP-D from pigs exhibits distinct anti-IAV properties and has potential as a prophylactic and/or therapeutic antiviral agent to protect humans against viral infections by IAV and other RNA viruses as COVID-19. The SARS-CoV infects host cells with its surface glycosylated spike-protein (S-protein) and S-protein within the alveoli is recognized by SP-D, allowing the regulation of pulmonary inflammation³⁰.

C. Innate Immune Soluble Proteins as Protectors Against Inflammation.

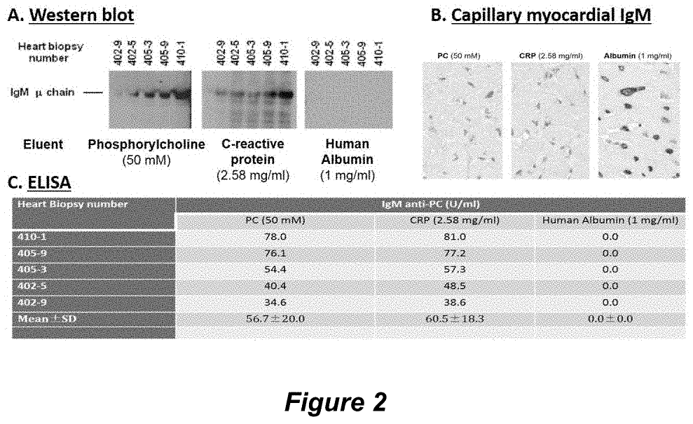

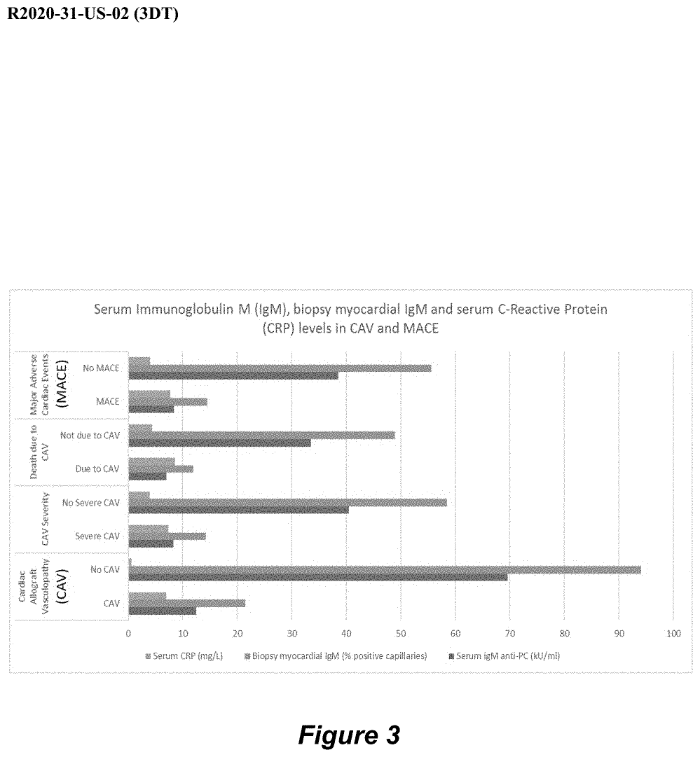

Pattern recognition innate immune collectins surfactant protein D (SP-D) and SP-A, and natural immunoglobulin M (IgM) are soluble proteins that enhance late apoptotic cell clearance in the lungs by alveolar macrophages. Collectins could be considered as specialized ‘antibodies of the innate immune system’³⁵. Innate and natural immune proteins SP-D, SP-A and IgM can interact with each other on late apoptotic cells and increase their clearance (see FIG. 1)³⁶. SP-D:IgM interactions occurring on late apoptotic cells appear not to interfere with the clearance of these cells³⁶, and the SP-D:IgM ratio may also modulate apoptotic cell clearance⁶. Alveolar macrophages internalize IgM- and SP-D-coated late apoptotic cells more effectively than uncoated cells, in vivo. Like antibodies, collectins also recognize and aggregate various microbes and other target molecules and enhance their clearance by phagocytes³⁵. Collectins, IgM and other soluble proteins are involved in recognizing and clearing dying cells⁶. It is becoming clear that collectins such as SP-A and SP-D play an important role in recognizing apoptotic cells and nucleic acids, and their clearance³⁵, and that IgM natural antibodies (NAbs) particularly promote clearance of small size particles³⁷. Probably about 80% of all NAbs circulating in the human body are natural IgMs, which are also the best-known immunoglobulins³⁸. NAbs provide the first line of defense against infection³⁹. NAbs have been shown to provide protection against influenza and other viruses. In addition to NAbs to the aforementioned organisms, B-1 cells produce “induced” antibody responses against influenza virus³⁹. C-reactive protein may also be able to enhance apoptotic cell clearance while minimizing inflammation⁶, especially in patients with severe alveolar damage and pneumonia. The cardiac data disclosed herein showed that IgM NAbs are protective and most probably recognize damaged/apoptotic endothelial cells within transplanted hearts (FIG. 2), avoiding inflammation, and reducing development and progression of cardiac allograft vasculopathy (atherosclerosis-like lesions) and major adverse cardiac events (FIG. 3). These findings are consistent with the protective function exercised by SP-D, SP-A and IgM NAbs enhancing the clearance of viruses, bacteria and apoptotic cells by lung alveolar macrophages⁶.

A determination of the effects of surfactant, SP-D and IgM NAbs upon alveolar damage, solely or in combination, in a coronavirus infected swine model, is discussed herein. As noted above, surfactant and its components SP-D and SP-A participate in the clearance of viral particles and the removal of apoptotic cells reducing inflammation^{3,4}. In recent reports, several strategies have been described for boosting natural IgM levels. Following splenectomy or thermal injury, patients often develop a selective loss of circulating IgM and display an associated heightened susceptibility to certain types of infections⁴¹. The use of two ways of treatment, namely Pneumococcal vaccination and i.v. administration of IgM Nabs, is discussed herein. Pneumococcal vaccination exploits the molecular mimicry among the PC moieties of microbial cell-wall polysaccharide, unfractionated OxLDL, and apoptotic cells⁴¹. As normal human plasma contains a substantial amount of IgM NAbs, it may be practical and economically viable to harness therapeutic potential of these IgM through the generation of therapeutic preparations in a manner analogous to intravenous immunoglobulins (IVIg) that is now extensively used for the treatment of a wide range of pathological conditions. By virtue of the diverse repertoire of immunoglobulins that possess a wide spectrum of antibacterial and antiviral specificities, IVIg provides antimicrobial efficacy independently of pathogen resistance and represents a promising alternative strategy for the treatment of diseases for which a specific therapy is not yet available. Controlled trials, particularly with viral diseases and certain defined septic subgroups where IVIg represents a promising but unproven treatment, are imperative⁴². Indeed, an IgM-enriched Ig preparation, pentaglobin, contains 12% IgM, and this has been successfully used for treating infections associated with sepsis in patients, as well as transplant rejection, and for certain inflammatory conditions in experimental models⁴¹.

This disclosure includes disclosure of the effects of surfactant, SP-D, surfactant plus IgM NAbs, SP-D plus IgM, surfactant plus SP-D plus IgM and no treatment in porcine respiratory coronavirus (PRCV)-infected pigs. Inoculated pigs will develop severe respiratory disease, and administration of surfactant, SP-D, surfactant and SP-D in combination with IgM NAbs, and administration of surfactant plus SP-D plus IgM NAbs will ameliorate the disease and inflammation associated with the disease, while clinical signs and markers of inflammation in the control group will be minimal or absent.

As such, the current disclosure includes treatment of novel coronaviruses with surfactant, surfactant proteins A and D, and IgM reducing inflammation and damage to alveoli.

Dosage of Surfactant (Curosurf) can be given in a total maximal dose of 400 mg/kg weight^{31,33}. Native pig SP-D (NpSP-D) can be isolated from pig lungs as described⁴⁶. For this purpose, six months old surplus pigs are used that were euthanized for other purposes. In short, NpSP-D are isolated from lung lavage by affinity purification method using Mannan-sepharose beads. After elution from the beads with EDTA-containing buffer, NpSP-D is purified using gel filtration chromatography⁴⁷. In one embodiment SP-D administered comprises 0.3 mg in 1 ml PBS based on previous experiments in mice⁴⁸. An intravenous infusion of 250 mg/kg (5 mL/kg) per day of IgM-enriched immunoglobulins (Pentaglobin)⁴⁹ can also be administered.

In an exemplary embodiment of a method of use of the invention, a mammalian patient suffering from a respiratory virus, such as a human, is administered surfactant. The surfactant may comprise surfactant proteins SP-D or SP-A, or a combination of the two. In an embodiment, only one surfactant protein is used for treatment. An alternate embodiment comprises both surfactant proteins. In another embodiment, the surfactant proteins are exogenous and preferably derived from a porcine source like pigs. Either SP-D or SP-A may be porcine derived, and preferably both are porcine derived where used in combination.

The surfactant is introduced into the airway of the patient, such as in an aerosol format, where it is inhaled to contact and coat the alveoli. The administration of surfactant proteins may be performed preventatively, before the patient is put on a ventilator, or after ventilation.

IgM can be administered intravenously in combination with the administration of surfactant proteins, as described above. In a preferred embodiment, the IgM is administered in conjunction with the surfactant proteins, SP-D or SP-A or a combination of the two. However, it is within the scope of this invention that IgM is administered alone.

Diseases treated can include respiratory viruses infecting the lung tissue, such as influenza, severe acute respiratory syndrome caused by coronaviruses, or any other diseases where

While various embodiments of methods of treating patients the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

1. Nkadi P O, Merritt T A, Pillers D-A M. An overview of pulmonary surfactant in the neonate: Genetics, metabolism, and the role of surfactant in health and disease. Mol Genet Metab 2009; 97: 95-101.
2. Voelker D R, Numata M. Phospholipid regulation of innate immunity and respiratory viral infection. J Biol Chem 2019; 294: 4282-4289.
3. Haagsman H P, Herias V, van Eijk M. Surfactant phospholipids and proteins in lung defence. Acta Pharmacol Sin 2003; 24: 1301-1303.
4. Hillaire M L B, Haagsman H P, Osterhaus A D M E, Rimmelzwaan G F, van Eijk M. Pulmonary surfactant protein D in first-line innate defence against influenza A virus infections. J Innate Immun 2013; 5: 197-208.
5. Al-Qahtani A A, Murugaiah V, Bashir H A, Pathan A A, Abozaid S M, Makarov E, Nal-Rogier B, Kishore U, Al-Ahdal M N. Full-length human surfactant protein A inhibits influenza A virus infection of A549 lung epithelial cells: A recombinant form containing neck and lectin domains promotes infectivity. Immunobiol 2019; 224: 408-418.
6. Litvack M L, Palaniyar N. Soluble innate immune pattern-recognition proteins for clearing dying cells and cellular components: implications on exacerbating or resolving inflammation. Innate Immunity 2010; 16: 191-200.
7. Qian Z, Travanty E A, Oko L, Edeen K, Berglund A, Wang J, Ito Y, Holmes K V, Mason R J. Innate immune response of human alveolar type II cells infected with severe acute respiratory syndrome-coronavirus. Am J Respir Cell Mol Biol 2013: 48: 742-748.
8. Jonsdottir H R, Dijkman R. Coronavirus and the human airway: a universal system for virus-host interaction studies. Virology Journal 2016; 13: 24.
9. Yan R, Zhang Y, Li Y, Xia L, Guo Y, Zhou Q. Structural basis for the recognition of the SARS-CoV-2 by full-length human ACE2. Science 2020; 10.1126/science. Abb 2762.
10. Baig A M, Khaleeq A, Ali U, Syeda H. Evidence of the COVID-19 virus targeting the CNS: Tissue distribution, host-virus interaction, and proposed neurotropic mechanisms. ACS Chem Neurosci 2020. https://dx.doi.org/10.1021/acschemneuro.0c00122.
11. Cascella M, Rajnik M, Cuomo A, Dulebohn S C, Di Napoli R. Features, evaluation and treatment coronavirus (COVID-19). StatPearls [Internet] Treasure Island (FL): StatPearls Publishing; 2020. 2020 Mar. 8.
12. Weiss S R, Leibowitz J L. Coronavirus pathogenesis. Adv Virus Res 2011; 81:85-164
13. Wu D, Wu T, Liu Q, Yang Z. The SARS-CoV-2 outbreak: What we know. Int J Infect Dis 2020. pii: 51201-9712(20)30123-5. doi: 10.1016/j.ijid.2020.03.004.
14. Yang D, Leibowitz J L. The structure and functions of coronavirus genomic 3′ and 5′ ends. Virus Res. 2015; 206: 120-133.
15. Drosten C, Gunther S, Preiser W. Identification of a Novel Coronavirus in Patients with Severe Acute Respiratory Syndrome. N Engl J Med 2020; 348:1967-76.
16. Zaki A M, van Boheemen S, Bestebroer T M, Osterhaus A D M E, Fouchier R A M. Isolation of a novel coronavirus from a man with pneumonia in Saudi Arabia. N Engl J Med. 2012; 367: 1814-1820.
17. Zhu N, Zhang D, Wang W, et al. A Novel Coronavirus from Patients with Pneumonia in China, 2019. N Engl J Med 2020; 382:727-33.
18. Cui J, Li F, Shi Z. Origin and evolution of pathogenic coronaviruses. Nat Rev Microbiol 2019; 17:181-92.
19. Li X, Wang W, Zhao X, Zai J, Zhao Q, Li Y, Chaillon A. Transmission dynamics and evolutionary history of 2019-nCoV. J Med Virol 2020; 92: 501-511. The Lancet Infectious Diseases. Challenges of coronavirus disease 2019. Lancet Infect Dis 2020; 20: 261. doi: 10.1016/S1473-3099(20)30072-4.
20. Hui D S C, Zumla A. Severe acute respiratory syndrome: historical, epidemiologic, and clinical features. Infect Dis Clin North Am 2019; 33: 869-89.
21. Azhar E I, Hui D S C, Memish Z A, Drosten C, Zumla A. The Middle East respiratory syndrome (MERS). Infect Dis Clin North Am 2019; 33: 891-905.
22. Huang C, Wang Y, Li X. Clinical features of patients infected with 2019 coronavirus in Wuhan, China. Lancet 2020; published online January 24. https://doi.org/10.1016/50140-6736(20)30183-5.
23. Li G, Fan Y, Lai Y, et al. Coronavirus infections and immune responses. J Med Virol 2020; published online January 25. DOI:10.1002/jmv.25685.
24. Channappanavar R, Perlman S. Pathogenic human coronavirus infections: causes and consequences of cytokine storm and immunopathology. Semin Immunopathol 2017; 39: 529-39.
25. Zumla A, Hui D S, Azhar E I, Memish Z A, Maeurer M. Reducing mortality from 2019-nCoV: host-directed therapies should be an option. The Lancet 2020; 395: e35-e36.
26. Batawi S, Tarazan N, Al-Raddadi R, et al. Quality of life reported by survivors after hospitalization for Middle East respiratory syndrome (MERS). Health Qual Life Outcomes 2019; 17: 101.
27. Ngai J C, Ko F W, Ng S S, To K W, Tong M, Hui D S. The long-term impact of severe acute respiratory syndrome on pulmonary function, exercise capacity and health status. Respirology 2010; 15: 543-50
28. The Lancet Infectious Diseases. Challenges of coronavirus disease 2019. The Lancet Infectious Diseases 2020; 20: 261. https://doi.org/10.1016/S1473-3099:30072-4.
29. World Health Organization (WHO). Coronavirus disease 2019 (COVID-19) Situation Report—95. https://www.who.int/docs/default-source/coronaviruse/situation-reports/20200424-sitrep-95-covid-19.pdf?sfvrsn=e8065831_4.
30. Leth-Larsen R, Zhong F, Chow V T K, Holmskov U, Lu J. The SARS coronavirus spike glycoprotein is selectively recognized by lung surfactant protein D and activates macrophages. Immunobiol 2007; 212: 201-211.
31. Nouraeyan N, Lambrinakos-Raymond A, Leone M, Sant'Anna G. Surfactant administration in neonates: A review of delivery methods. Can J Respir Ther 2014; 50: 91-95.
32. Nkadi P O, Merritt T A, Pillers D-A M. An overview of pulmonary surfactant in the neonate: Genetics, metabolism, and the role of surfactant in health and disease. Mol Genet Metab 2009; 97: 95-101.
33. Polin R A, Carlo W A; Committee on Fetus and Newborn; American Academy of Pediatrics. Surfactant replacement therapy for preterm and term neonates with respiratory distress. Pediatrics 2014; 133: 156-63.
34. Engle W A; American Academy of Pediatrics Committee on Fetus and Newborn. Surfactant-replacement therapy for respiratory distress in the preterm and term neonate. Pediatrics. 2008; 121: 419-432.
35. Palaniyar N. Antibody equivalent molecules of the innate immune system: parallels between innate and adaptive immune proteins. Innate Immunity 2010; 16: 131-137.
36. Litvack M L, Djiadeu P, Renganathan S D S, Sy S, Post M, Palaniyar N. Natural IgM and innate immune collectin S P-D bind to late apoptotic cells and enhance their clearance by alveolar macrophages in vivo. Mol Immunol 2010; 48: 37-47.
37. Litvack M L, Post M, Palaniyar N. IgM promotes the clearance of small particles and apoptotic microparticles by macrophages. PLoS ONE 2011; 6: e17223.
38. Palma J, Tokarz-Deptula B, Deptula J, Deptula W. Natural antibodies—facts known and unknown. Centr Eur J Immunol 2018; 43: 466-475.
39. Holodick N E, Rodriguez-Zhurbenko N, Hernandez A M. Defining natural antibodies. Front Immunol 2017; 8: 872.
40. Wang J, Oberley-Deegan R, Wang S, Nikrad M, Funk C J, Hartshorn K L, Mason R J. Differentiated human alveolar type II cells secrete antiviral IL-29 (IFN-λ1) in response to influenza A infection. J Immunol 2009; 182: 1296-1304.
41. Kaveri S V, Silverman G J, Bayry J. Natural IgM in immune equilibrium and harnessing their therapeutic potential. J Immunol 2012; 188: 939-945.
42. Bayry J, Lacroix-Desmazes S, Kazatchkine M D, Kaveri S V. Intravenous immunoglobulin for infectious diseases: back to the pre-antibiotic and passive prophylaxis era? Trends Pharmacol Sci 2004; 25: 306-310.
43. Van Reeth K, Pensaert M B. Porcine respiratory coronavirus-mediated interference against influenza virus replication in the respiratory tract of feeder pigs. Am J Vet Res 1994; 55, 1275-1281.
44. Atanasova K, van Gucht S, Barbé F, Duchateau L, van Reeth K. Lipoteichoic acid from Staphylococcus aureus exacerbates respiratory disease in porcine respiratory coronavirus-infected pigs. The Veterinary Journal 2011; 188: 210-215.
45. Van Gucht S, Atanasova K, Barbé F, Cox E, Pensaert M, Van Reeth K. Effect of porcine respiratory coronavirus infection on lipopolysaccharide recognition proteins and haptoglobin levels in the lungs. Microbes Infect 2006; 8: 1492-1501.
46. van Eijk M, van de Lest C H A, Batenburg J J, Vaandrager A B, Meschi J, Hartshorn K L, van Golde L M G, Haagsman H P. Porcine surfactant protein D is N-glycosylated in its carbohydrate recognition domain and is assembled into differently charged oligomers. Am J Respir Cell Mol Biol 2002; 26: 739-747.
47. Hillaire M L B, van Eijk M, van Trierum S E, van Riel D, Saelens X, Romijn R A, Hemrika W, Fouchier R A M, Kuiken T, Osterhaus A D M E, Haagsman H P, Rimmelzwaan G F. Assessment of the antiviral properties of recombinant porcine S P-D against various influenza A viruses in vitro. PLoS ONE 2011; 6: e25005.
48. Madan T, Kishore U, Singh M, Strong P, Hussain E M, Reid K B M, Sarma P U. Protective role of lung surfactant protein D in a murine model of invasive pulmonary aspergillosis. Infect Immun 2001; 69: 2728-2731.
49. Domizi R, Adrario E, Damiani E, Scorcella C, Carsetti A, Giaccaglia P, Casarotta E, Gabbanelli V, Pantanetti S, Lamura E, Ciucani S, Donati A. IgM-enriched immunoglobulins (Pentaglobin) may improve the microcirculation in sepsis: a pilot randomized trial. Ann Intensive Care 2019; 9: 135.

1. A method of treating a mammalian patient infected with a respiratory virus comprising the step of administering a therapeutically effective amount of surfactant protein.

2. The method of claim 1 wherein the surfactant protein comprises surfactant protein D (SP-D).

3. The method of claim 1 wherein the surfactant protein comprises surfactant protein A (SP-A).

4. The method of claim 1 wherein the surfactant protein comprises a combination of SP-D and SP-A.

5. The method of claim 2 wherein the SP-D is exogenously derived.

6. The method of claim 5 wherein the SP-D is porcine derived.

7. The method of claim 1 wherein the surfactant protein is administered by inhalation.

8. The method of claim 7 further comprising the step of administering a therapeutically effective amount of Immunoglobulin M natural antibodies (IgM NAbs).

9. The method of claim 8 wherein the therapeutically effective amount of IgM NAbs is administered intravenously.

10. A method of treating a human patient infected with a severe acute respiratory syndrome coronavirus or a variant thereof, comprising the step of administering a therapeutically effective amount of surfactant and porcine SP-D and SPD-A.

11. The method of claim 10 further comprising the step of administering a therapeutically effective amount of IgM NAbs.

12. The method of claim 11 wherein the therapeutically effective amount of surfactant is introduced into the airway.

13. The method of claim 11 wherein the therapeutically effective amount of surfactant is in aerosol form.

14. The method of claim 13 wherein the therapeutically effective amount of IgM NAabs is introduced intravenously.

15. A method of treating a human patient infected with a severe acute respiratory syndrome coronavirus or a variant thereof, comprising the step of administering a therapeutically effective amount of SP-D.

16. The method of claim 15 further comprising the step of administering a therapeutically effective amount of IgM NAbs.

17. The method of claim 16 further comprising the step of administering a therapeutically effective amount of SP-A

18. The method of claim 16 wherein the SP-D is porcine derived.

19. The method of claim 16 further comprising the step of administering a therapeutically effective amount of surfactant.

20. The method of claim 15 wherein the severe acute respiratory syndrome coronavirus is SARS-CoV-2.
